(19) United States
(12) Patent Application Publication
(10) Pub. No.: US 2025/0115945 A1
(43) Pub. Date: Apr. 10, 2025

(54) CELL-FREE TRNA FRAGMENT-BASED TR-FRET SCREENING AND COMPETITION ASSAY FOR INHIBITORS OF HUMAN TRNA MODIFICATION ENZYMES USEFUL IN THE PREVENTION OR TREATMENT OF TRNA MODIFICATION RELATED CONDITIONS

(71) Applicant: GESVAL S.A., Liege (BE)

(72) Inventors: Pierre CLOSE, Liege (BE); Francesca RAPINO, Liege (BE); Alain CHARIOT, Neupre (BE)

(21) Appl. No.: 18/291,332

(22) PCT Filed: Jul. 16, 2022
(86) PCT No.: PCT/EP2022/069969

§ 371 Date: Jan. 23, 2024

(30) Foreign Application Priority Data
Aug. 13, 2021 (BE) BE2021/5646

(51) Int. Cl. C12Q 1/48; C12Q 1/6806; C12Q 1/6818; G01N 33/542

(52) U.S. Cl. CPC C12Q 1/48; C12Q 1/6806; C12Q 1/6818; G01N 33/542

(57) ABSTRACT

A Cell-free tRNA fragment-based FRET screening and competition assay for inhibitors of human tRNA modification enzymes useful in the prevention or treatment of tRNA modification-related conditions is provided.

### BACKGROUND

The availability of simple yet effective high throughput screening methods is fundamental in drug discovery.

One important screening method is the fluorescence resonance energy transfer (FRET). FRET is also referred to as Forster resonance energy transfer, resonance energy transfer or electronic energy transfer. FRET describes the energy transfer between two light sensitive molecules, so called chromophores. A donor chromophore in its electronic excited states transfers energy to an acceptor chromophore through nonradiative dipole-dipole coupling. The efficiency of this energy transfer is inversely correlated to the distance between donor and acceptor. Thus, FRET is sensitive to small distance changes. In biology, FRET efficiency is used to determine if two fluorophores are within a certain distances.

FRET is often used to detect and track interactions between proteins. Additionally, FRET can be used to measure distances between domains in a single protein by tagging different regions of the protein with fluorophores and measuring emission to determine distance.

This provides information about protein conformation, including secondary structures and protein folding.

Time-resolved fluorescence resonance energy combines the low background aspect of TRF with the homogeneous assay format of FRET. The resulting assay provides an increase in flexibility, reliability and sensitivity in addition to higher throughput and fewer false positive or false negative results. FRET involves two fluorophores, a donor and an acceptor. Excitation of the donor by an energy source (e.g. flash lamp or laser) produces an energy transfer to the acceptor, if the two are within a given proximity to each other. The acceptor in turn emits light at its characteristic wavelength.

However, when TR-FRET screening large libraries of, for example 20 000 compounds, it is key to further distinguish false positive results from promising leads.

### PRIOR ART

FRET assays have already been described to screen for t-RNA inhibitors that are useful as antibiotics against bacterial propagation.

Goldman (US2015118678A1) discloses a method for identifying test compounds with antimicrobial activity by mixing an elongation factor with GTP and an aminoacylated transfer RNA in the presence of said test compound. The elongation factor is operably linked to a first energy transfer pair member. The aminoacylated tRNA is operably linked to a second energy transfer pair member. The first energy transfer pair member and the second energy transfer pair member form a fluorescence resonance energy transfer pair.

The level of fluorescence in the presence of the test compound is compared to the level of fluorescence in the absence of the test compound or the level of fluorescence in the presence of a control compound. The difference in fluorescence indicates that the test compound possesses antimicrobial activity. However, no credible disclosure relating to the identification of inhibitors of human tRNA modification enzymes is provided. Production of full-length t-RNA is very expensive and thus the disclosed assay involves high manufacturing costs.

Guenther (US2008199870A1) discloses compositions and methods for identifying inhibitors of interactions between an RNA and a target molecule. A mixture comprising a tRNA fragment with a modified nucleotide, a target molecule capable of binding to the tRNA fragment, and a test compound is incubated under conditions that allow binding of the tRNA and the target molecule in the absence of the test compound. Assays can then be performed that detect whether or not the test compound inhibits the binding of the tRNA molecule and the target molecule. High throughput assays are also described. However, no credible disclosure relating to the identification of inhibitors of human tRNA modification enzymes is provided.

WO 2012/135416 (Guenther) describes methods for inhibiting S. aureus propagation, and screening for compounds that inhibit S. aureus propagation, are described. In particular, this document describes a method of inhibiting S. aureus propagation through either inhibiting or stabilizing ribosomal binding of a specific S. aureus tRNA in the S. aureus by an amount sufficient to inhibit S. aureus protein expression. However, no credible disclosure relating to the identification of inhibitors of human tRNA modification enzymes is provided.

Bhatt et al, Engineered EF-Tu and tRNA-Based FRET Screening Assay to Find Inhibitors of Protein Synthesis in Bacteria, ASSAY and Drug Development Technologies, VOL. 16 NO. 4 2018, DOI: 10.1089/adt.2018.843 describe a HTS FRET assay for the identification of inhibitors of ternary complex formation in bacteria in a search for new antibiotics. However, no credible disclosure relating to the identification of inhibitors of human tRNA modification enzymes is provided.

Therefore, there remains a need for an efficient, cost-effective screening of inhibitors of human tRNA modification enzymes that are useful in the treatment or prevention of a tRNA-related condition, in particular of cancer.

### Short Description of the Invention

The inventors have surprisingly found that a cell-free tRNA fragment based FRET screening and competition assay can be cost-efficiently used in high throughput screenings of large compound libraries to identify inhibitors of human tRNA modification enzymes that may be useful in the treatment of tRNA modification related conditions such as cancer. In a preferred embodiment, the tRNA inhibitors are able to interfere with the binding of Elongator complex protein 3 to tRNAs.

Accordingly, a first aspect of the invention is a cell-free HTS assay for detecting inhibitors of human tRNA modification enzymes:
a, wherein a cell-free TR-FRET screening assay is conducted by
   contacting in a screening reaction mixture
      a tRNA fragment as a substrate for an human tRNA modification enzyme
      a purified human tRNA modification enzyme and
      a candidate inhibitor compound,
      wherein the substrate is labeled with a donor fluorophore and
      wherein the purified human tRNA modification enzyme is labeled with a acceptor fluorophore that is able to absorb the energy emitted by the donor fluorophore; or
      the substrate is labeled with an acceptor fluorophore moiety and the purified human tRNA modification enzyme is labeled with a donor fluorophore, and
   exposing the screening reaction mixture to a light source;
   measuring the fluorescence of the screening reaction mixture by a fluorescence emission detector, and
   selecting the candidate inhibitors that reduce the TR-FRET signal;
b, wherein a cell-free TR-FRET competition assay is carried out to screen out false positives of the TR-FRET-based screening assay of step a.

In another aspect of the cell-free HTS assay, the tRNA fragment is a fragment of the anticodon loop of the tRNA.

In another aspect of the cell-free HTS assay, the tRNA fragment comprises between 15 and 21 bases.

In another aspect of the cell-free HTS assay, the tRNA fragment comprises 19 bases. In another aspect of the cell-free HTS assay, the human tRNA modification enzyme is an enzyme interacting with the anticodon loop, the D-loop or the T-loop.

In another aspect of the cell-free HTS assay, the human tRNA modification enzyme is an anticodon binding enzyme.

In another aspect of the cell-free HTS assay, the human tRNA modification enzyme is an non active recombinant form of the enzyme.

In another aspect of the cell-free HTS assay, the human tRNA modification enzyme is ELP3.

In another aspect of the cell-free HTS assay, the human tRNA modification enzyme is GST/HIS-tagged.

In another aspect of the cell-free HTS assay, the tRNA fragment is labeled with dye or biotinylated either at the 5′ or at the 3′ end.

In another aspect of the cell-free HTS assay, the tRNA fragment is a 19 oligomer with a biotinylated 3′ end.

In another aspect of the cell-free HTS assay, the TR-FRET competition assay comprises the steps of
a. providing a fluorophore tagged protein susceptible of replacing the fluorescence tagged tRNA modification enzyme tRNA interaction
b. contacting the fluorophore tagged protein with the candidate inhibitors identified in the TR-FRET screening assay in a competition reaction mix;
c. exposing the competition reaction mixture to a light source;
d. measuring the fluorescence of the competition reaction mixture by a fluorescence emission detector;
e. selecting the candidate inhibitors that are not modulating the TR-FRET signal.

In another aspect of the cell-free HTS assay, the fluorescence tagged protein is a biotinylated tagged protein that replaces the tagged tRNA modification-enzyme biotinylated tRNA antistem loop interaction.

Another aspect of the invention is the use of the cell-free HTS assay of the invention for the detection of inhibitors of human tRNA modification enzymes in the prevention or treatment of tRNA modification related conditions, in particular cancer or metastatic cancer.

Another aspect of the present invention is a cell-free HTS system for the detection of an inhibitor of a tRNA modification enzyme configured to carry out the assay of the present invention.

### DETAILED DESCRIPTION OF THE INVENTION

tRNA

A transfer ribonucleic acid, hereinafter referred to as tRNA, is a molecule composed of typically 70 to 90 ribonucleotides. It serves as the link between the nucleotide sequence of nucleic acids and the amino acid sequence of proteins. Its primary function is carrying a specific amino acid to the ribosome as directed by the three-nucleotide codon sequence in messenger RNA, the so-called mRNA.

61 mRNA codons code for 20 amino acids. Different genes encode sequence variants of tRNAs recognizing the same mRNA codon. Consequently, tRNAs repertoires are extremely heterogeneous among tissues and cellular populations. The tissue specificity of tRNA pools is furthermore increased by the presence of a variety of tRNA modification patterns.

tRNA Modification in Cancer

tRNA diversity has a key role in proteome rewiring in cancer. For example, breast cancer-derived cells expressed significantly increased amounts of specific tRNAs compared to healthy breast lines. This specific overexpression was linked with breast metastatic potential.

The present inventors already demonstrated that certain modified tRNAs are responsible for the decoding fidelity of codons enriched in cancers. These modified tRNAs are necessary to correctly establish onco-proteomes, Sources: F. Rapino et al., “Codon-specific translation reprogramming promotes resistance to targeted therapy,” Nature, 2018. F. Rapino, S. Delaunay, Z. Zhou, A. Chariot, and P. Close, “tRNA Modification: Is Cancer Having a Wobble?,” Trends in Cancer. 2017.

Labeled tRNA Fragments

The tRNA for the present assay are fragments of tRNAs. In a preferred embodiment, the tRNA is a biotinylated tRNA fragment of 15 to 21 bases. In a particularly preferred embodiment, the tRNA fragment is a 19 oligomer and even more preferably a 19 oligomer of the following sequences:
5′-gacggccuUUCacgccguc-3′

The tRNA fragments are labeled with Dye or biotinylated either at the 5′ or at thee 3′ end. In a preferred embodiment, the tRNA fragment is a 19 oligomer sequence that is biotinylated at its 3′ end.

tRNA Modification Enzyme

In a preferred embodiment, the tRNA modification enzyme is the Elongator complex protein 3, also named KAT9. ELP3 has the Uniprot IP Q9H9T3 1. ELP3 is encoded by the ELP3 gene in humans and has the Gene ID 55140. ELP3 has been initially described as a protein involved in transcriptional elongation. ELP3 consists of 547 amino acids and has a molecular weight of 62.259 Dalton.

Any substantially purified tRNA modification enzyme is suitable for the assay of the present invention. The enzyme may be active or inactive for the purposes of identifying suitable inhibitors of tRNA modification enzymes.

### Cell-Free TR-FRET Screening Assay

The cell-free TR-FRET assay may be conducted by
contacting in a screening reaction mixture
   a tRNA fragment as a substrate for a human tRNA modification enzyme
   a purified human tRNA modification enzyme and
   a candidate inhibitor compound,
   wherein the substrate is labeled with a donor fluorophore and
   wherein the purified human tRNA modification enzyme is labeled with a acceptor fluorophore that is able to absorb the energy emitted by the donor fluorophore; or
   the substrate is labeled with an acceptor fluorophore and the purified human tRNA modification enzyme is labeled with a donor fluorophore, and
exposing the screening reaction mixture to a light source;
measuring the fluorescence of the screening reaction mixture by a fluorescence emission detector, and
selecting the candidate inhibitors that reduce the TR-FRET signal;

Inhibitors of human tRNA modification enzymes reduce its binding on the labelled tRNA and thus will reduce the fluorescence emission of the acceptor fluorophore at the wavelength of the donor fluorophore.

In a preferred embodiment, the tRNA fragment is incubated in an annealing buffer comprising 20 mM Hepes pH 7.5, 50 mM KCl and 50 mM NaCl, to which preferably 5 mM MgCl2 are added.

In another embodiment, the screening reaction mixture comprises a screening buffer consisting of:
10 mM to 30 mM Bis-Tris pH 5.5.
20 mM to 100 mM NaCl
2.5 mM to 10 mM MgCl2
0.001% BSA
0.001% to 0.05% Tween
0.25 mM to 1 mM DTT
0.5 mM to 2 mM EDTA

### Concentration

The tRNA modification enzyme is usually present in a concentration from 0.5 nM to 70 nM.

The tRNA fragment is usually present in a concentration from 5 nM to 100 nM.

The detection tools are usually present in a concentration from 1 nM to 20 nM for GST and 1 nM to 90 nM for SA.

### Buffer

In one embodiment, the buffer comprises one or more of following components:

| Reagent | Tolerable range | alternative |
|---|---|---|
| MES | pH 5.5 | |
| Bis Tris | pH 5.5-6.5 | MES pH 5.5, PIPES |
| | | pH 6, HEPES pH |
| | | 6.5 |
| PIPES | pH 6-6.5 | |
| NaP | pH 6.0-6.5 | |
| Hepes | pH 6.5 | |
| NaCl | 50-100 mM | |
| KCI | 100 mM | |
| MgC12 | 0-5 mM | In combination with |
| | | 100 mM NaCl |
| Tween-20 | 0.001-0.1% | BRijL23 (0.001%) |
| Tween-80 | 0.01% | |
| Igepal-CA630 | 0.01% | |
| Triton X-100 | 0.01% | |
| Pluronic F-127 | 0.1%-0.01% | |
| Brij-L23 | 0.001%-0.01% | |
| Faf-BSA | 0.003%-0.03% | Faf-pf-hs-BSA |
| | | (0.002%) |
| EDTA | >3.16 mM | |
| EGTA | >3.16 mM | |
| DTT | <5 mM | |
| ZnCl2 | <100 micromolar | |
| NiSO4 | <100 micromolar | |
| CuSO4 | <1 micromolar | |
| PdCI2 | <1 micromolar | |

### Cell-Free TR-FRET Competition Assay

The TR-FRET competition assay of the present invention comprises the steps of
a. Providing a fluorophore tagged protein susceptible of replacing the interaction between the fluorescence tagged tRNA modification enzyme and the tRNA
b. Contacting the fluorophore tagged protein with the candidate inhibitors identified in the TR-FRET screening assay of the present invention in a competition reaction mix;
c. exposing the competition reaction mixture to a light source;
d. measuring the fluorescence of the competition reaction mixture by a fluorescence emission detector;
e. selecting the candidate inhibitors that are not modulating the TR-FRET signal.

In a preferred embodiment, the fluorophore tagged protein is a biotinylated tagged protein that replaces the tagged tRNA modification enzyme biotinylated tRNA antistem loop interaction. This step is key to identify false positive of the cell-free TR-FRET screening assay of the present invention.

In another embodiment, the tRNA modification enzyme is the U34-enzyme ELP3.

In another embodiment, the competition buffer consists of:
25 mM Bis-Tris pH 5.5
100 mM NaCl
2.5 mM MgCl2
0.001% faf-BSA
0.001% Tween-20
0.5 mM DTT
1 mM EDTA
   Indications: tRNA Modification-Related Condition

The present is assay may be used to detect inhibitors of a tRNA modification-related condition, in particular cancer or neurodegenerative diseases such as amyotrophic lateral sclerosis.

### Advantages

This cell-free TR-FRET-based competition assay is homogeneous, non-hazardous, high-throughput, and has high sensitivity allowing for low usage of material and low cost. Moreover, it is based on tRNA fragments and thus reduces both cost and complexity of the high throughput screening for drug discovery. The present cell-free TR-FRET-based competition assay does not require an active t-RNA modification enzyme to function. This may substantially reduce the costs and complexity of High Througput screening.

### SHORT DESCRIPTION OF THE DRAWINGS

FIG. 1a schematically shows a TR-FRET screening assay of the invention in an embodiment with ELP3 as the tRNA modification enzyme with GST/His tag and a donor or acceptor and a biotinylated tRNA fragment with a receptor or a donor. An ELP3 inhibitor reduces the high TR-FRET signal. FIG. 1b schematically shows a TR-FRET competition assay of the invention in an embodiment with ELP3 as the tRNA modification enzyme with GST tag and terbium as donor and labeled Streptonin as acceptor.

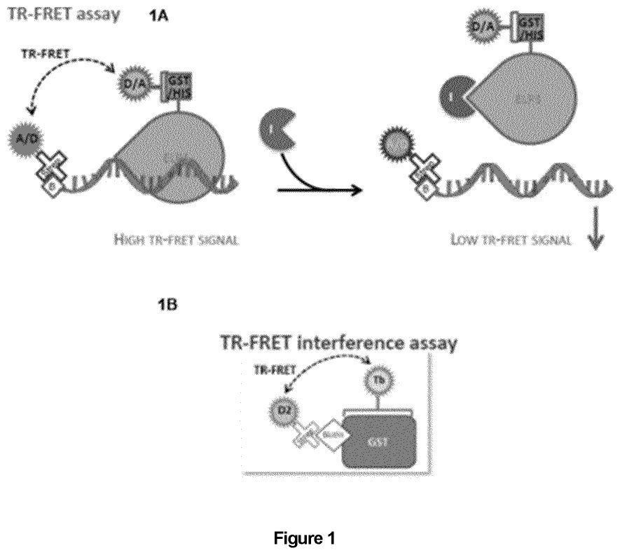

FIG. 2A shows the refolding of synthesized tRNA and FIG. 2B shows the TR-FRET screening assay of the present invention including optimized buffers for tRNA modification enzyme ELP3.

FIG. 3 shows an overview the steps of the TR-FRET competition assay of the present invention including optimized buffers for tRNA modification enzyme ELP3

### EXAMPLES—TR FRET BASED ELP3 TRNA SCREENING ASSAY

### GST/HIS Tagged ELP3 Proteins:

Following GST/HIS tagged ELP3 were used:

| Nº | Tag | Name | Host | Provider |
|---|---|---|---|---|
| 1 | HIS | ELP3 (AA 1 | Sf9 | Antibodies |
| | | 547) protein (His tag) | | Onine |
| 2 | GST | KAT9 (ELP3) Protein | Sf9 | Signal Chem |
| 3 | GST | Human Elp3 protein | Wheat germ | Novus |

### Anti-GST/HIS Detection Tools:

Following GST/HIS tagged detection tools were used:

| Nº | Tag | Name | Label | Provider |
|---|---|---|---|---|
| 1 | Anti-HIS | Mab Anti 6HIS d2 | D2 (Acceptor) | CisBio |
| 2 | Anti-HIS | Mab Anti 6HIS XL665 | XL665 (Acceptor) | Signal Chem |
| 3 | Anti-HIS | Mab Anti 6HIS Tb | Terbium (Donor) | Novus |
| | | cryptate | | |
| 4 | Anti-GST | Mab Anti 6HIS d2 | D2 (Acceptor) | CisBio |
| 5 | Anti-GST | Mab Anti 6HIS XL665 | XL665 (Acceptor) | Signal Chem |
| 6 | Anti-GST | Mab Anti 6HIS Tb | Terbium (Donor) | Novus |
| | | cryptate | | |

Synthesized tRNA Fragments

Unlabeled, biotinylated and day-labelled tRNA fragments were synthesized. Following tRNA fragment was used for the HTS assay.
5′-gacggccuUUCacgccguc-3′

### ELP3/tRNA TR-FRET Assay Configuration

Following combination was chosen for the ELP3/tRNA TR-FRET assay:

| Component | |
|---|---|
| ELP3 | GST-ELP3 |
| tRNA | tRNA UUC #3 |
| Label | 3′Biotin |
| Detection | GST-Tb/SA XL655 |

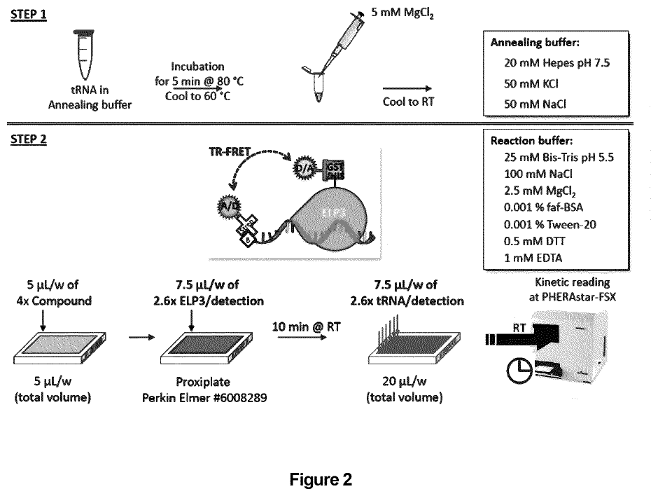

Re-Folding of tRNA-FIG. 2A

The synthesized tRNA was re-folded in a thermocycler using an annealing buffer consisting of 20 mM Hepes pH 7.5, 50 mM KCl, 50 mM NaCl. The mixture was incubated for 5 minutes at 80° C. and then cooled to 60° C. Then, 5 mM Mg Cl2 were added and the mixture was cooled to room temperature.

### Screening Assay—FIG. 2B

5 microliter of ELP3 Mix were put in each well of an 96 well assay plate (Proxiplate) commercially available from Perkin Elmer). Then, 5 microliter per well of tRNA Mix were added leading to a total well volume of 10 microliters. The mixture was incubated for 10 minutes at room temperature. Then, 10 microliters of detection mix were added per well leading to a final volume of 20 microliters/w. The mixture was then subjected to kinetic reading at PHERAstar-FSX at an excitation of 337 nanometres and an emission of 665 and 620 nanometres.

GST/HIS tagged ELP3 interacts with Biotin labelled tRNA. Consequently, the ELP3 tRNA complex brings Donor or Acceptor labelled anti GST/HIS antibody and Acceptor or Donor labelled streptavidin in close proximity.

Excitation of the Donor at 320 nm results in energy transfer to the acceptor and emission at a higher wavelength (665 nm) after a time delay (TR FRET signal). Thus, the interaction between ELP3 and tRNA oligo/tRNA is measured as increase of the TR FRET signal.

Inhibition of ELP3 tRNA interaction by a candidate inhibitor occurs by displacement of the tRNA from the ELP3 binding site and, consequently, leads to a decrease of the TR FRET signal.

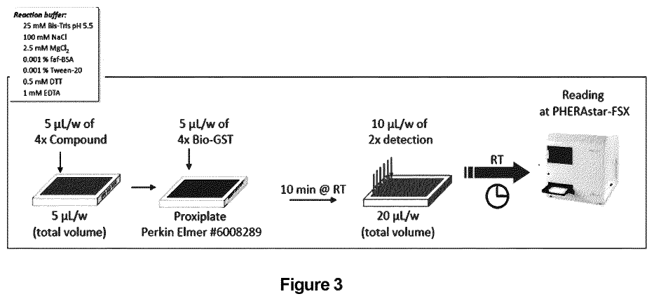

### Competition Assay-FIG. 3

The TR-FRET competition assay relies on biotinylated tagged protein that replaces the tagged TM-enzyme biotinylated tRNA antistem loop interaction. This step is key to identify false positive of the primary screen. Inhibitors are identified as non-modulating the FRET signal in this configuration.

The competition assay was optimized for the U34 enzyme ELP3. An overview of the TR-FRET competition assay is shown in FIG. 3.

TABLE 2
Translation of abbreviations and of the English expressions
used in the drawings:

| English expression | Translation |
|---|---|
| TR-FRET assay | |
| ELP3 | |
| High TR-FRET signal | |
| Low TR-FRET signal | |
| A/D | |
| D/A | |
| GST/HIS | |
| Strep | |
| B | |
| Annealing buffer | |
| Incubation | |
| For 5 min | |
| Cool to RT = Room temperature | |
| 5 mM MCl2 | |
| Step | |
| Compound | |
| Total volume | |
| detection | |
| Proxiplate perkin elmer #6008289 | |
| Kinetic reading | |
| Reaction buffer | |
| Reading at | |

The invention claimed is:

1. A cell-free HTS assay for detecting inhibitors of human tRNA modification enzymes:
a. wherein a cell-free TR-FRET screening assay is conducted by
contacting in a screening reaction mixture
a tRNA fragment as a substrate for an human tRNA modification enzyme
a purified human tRNA modification enzyme and
a candidate inhibitor compound,
wherein the substrate is labeled with a donor fluorophore and
wherein the purified human tRNA modification enzyme is labeled with a acceptor fluorophore that is able to absorb the energy emitted by the donor fluorophore; or
the substrate is labeled with an acceptor fluorophore and the purified human tRNA modification enzyme is labeled with a donor fluorophore, and
exposing the screening reaction mixture to a light source;
measuring the fluorescence of the screening reaction mixture by a fluorescence emission detector; and
selecting the candidate inhibitors that reduce the TR-FRET signal;
b. wherein a cell-free TR-FRET competition assay is carried out to screen out false positives of the TR-FRET-based screening assay of step a.

2. The cell-free HTS assay of claim 1, wherein the tRNA fragment is a fragment of the anticodon loop of the tRNA.

3. The cell-free assay of claim 1, wherein the tRNA fragment comprises between 15 and 21 bases.

4. The cell-free HTS assay of claim 1, wherein the tRNA fragment comprises 19 bases.

5. The cell-free HTS assay of claim 1, wherein the human tRNA modification enzyme is an enzyme interacting with the t-stem loop, the D-loop or the T-loop.

6. The cell-free HTS assay of claim 1, wherein the human tRNA modification enzyme is an anticodon binding enzyme.

7. The cell-free HTS assay of claim 1, wherein the human tRNA modification enzyme is ELP3.

8. The cell-free HTS assay of claim 1, wherein the human tRNA modification enzyme is an non active recombinant form of the enzyme.

9. The cell-free HTS assay claim 1, wherein the human tRNA modification enzyme is GST/HIS-tagged.

10. The cell-free HTS assay of claim 1, wherein the tRNA fragment is labeled with dye or biotinylated either at the 5′ or at the 3′ end.

11. The cell-free HTS assay of claim 1, wherein the tRNA fragment is a 19 oligomer with a biotinylated 3′ end.

12. The cell-free HTS assay of claim 1, wherein TR-FRET competition assay comprises the steps of
a. providing a fluorophore tagged protein susceptible of replacing the fluorescence tagged tRNA modification enzyme tRNA interaction;
b. contacting the fluorophore tagged protein with the candidate inhibitors identified in the TR-FRET screening assay in a competition reaction mix;
c. exposing the competition reaction mixture to a light source;
d. measuring the fluorescence of the competition reaction mixture by a fluorescence emission detector;
e. selecting the candidate inhibitors that are not modulating the TR-FRET signal.

13. The cell-free HTS assay of claim 1, wherein the fluorescence tagged protein is a biotinylated tagged protein that replaces the tagged tRNA modification-enzyme biotinylated tRNA antistem loop interaction.

14. (canceled)

15. (canceled)

16. A method for detecting inhibitors of human tRNA modification enzymes:
wherein a cell-free TR-FRET screening assay is conducted by
contacting in a screening reaction mixture
a tRNA fragment as a substrate for an human tRNA modification enzyme
a purified human tRNA modification enzyme and
a candidate inhibitor compound,
wherein the substrate is labeled with a donor fluorophore and wherein the purified human tRNA modification enzyme is labeled with a acceptor fluorophore that is able to absorb the energy emitted by the donor fluorophore; or
the substrate is labeled with an acceptor fluorophore and the purified human tRNA modification enzyme is labeled with a donor fluorophore, and
exposing the screening reaction mixture to a light source;
measuring the fluorescence of the screening reaction mixture by a fluorescence emission detector; and
selecting the candidate inhibitors that reduce the TR-FRET signal;
wherein a cell-free TR-FRET competition assay is carried out to screen out false positives of the TR-FRET-based screening assay of step a.

17. A cell-free HTS system for the detection of an inhibitor of a tRNA modification enzyme configured to carry out the assay of claim 1.
